(19) United States
(12) Patent Application Publication
(10) Pub. No.: US 2020/0110019 A1
(43) Pub. Date: Apr. 9, 2020

(54) Atmospheric pollution source mapping and tracking of pollutants by using air quality monitoring networks having high space-time resolution

(71) Applicant: Sense Square S.r.l.s., Salerno (IT)

(72) Inventors: Daniele Sofia, Pontecagnano Faiano (SA) (IT); Aristide Giuliano, Salerno (IT); Giovanni D'Acunto, Salerno (IT); Marco Polverino, Salerno (IT)

(21) Appl. No.: 16/618,080

(22) PCT Filed: Jun. 8, 2018
(86) PCT No.: PCT/IB2018/054162

§ 371 Date: Nov. 27, 2019

(30) Foreign Application Priority Data
Jun. 9, 2017 (IT) 102017000064056

(51) Int. Cl. G01N 15/06; G01W 1/06; G01N 1/22; G01N 33/00; H04W 4/38

(52) U.S. Cl. CPC G01N 15/06; G01W 1/06; G01W 2001/006; G01N 33/0016; H04W 4/38; G01N 1/2273

(57) ABSTRACT

A method and corresponding apparatus for monitoring air quality while allowing the real tracking of both gaseous and powder pollutants and the identification of the sources of pollution, includes the use of a network of monitoring stations mutually arranged at a maximum distance between one and three kilometers in a straight line, wherein each station is positioned at a vertex of a plurality of adjacent triangles or meshes into the geographic zone to be mapped, wherein each monitoring station is configured to pre-process the air data quality together with the weather data and to send it with its GPS coordinates to a central collection and processing computer, and wherein the data received by the single stations are classified, recorded and processed in real time using an algorithm configured to directly provide the positions of the pollution sources.

### SUMMARY OF THE INVENTION

The present invention relates to a method and respective apparatus for identifying and mapping air pollution sources using innovative station networks for monitoring air pollution concentrations and atmospheric parameters. Such monitoring networks consist of a minimum of three stations placed at a distance in a straight line from one another, capable of ensuring a high amount of data per unit of surface, within a single territory. Data on concentrations of pollutants and meteorological parameters is sent in real time. The particular structure of the measuring chamber of the monitoring station allows very accurate measurements in all weather conditions.

The computer processing of data obtained from air quality monitoring networks having high space and time resolution, performed with innovative algorithms which will be described below, allow to map the sources of pollution and identify those who are most responsible. This system also allows the real-time tracking of gaseous pollutants, thus allowing the delineation of preferential routes and stagnation zones.

### FIELD OF THE INVENTION

In urban areas, it is becoming increasingly common to find a series of substances in the atmosphere which can cause direct damage to human health. They are constantly produced by human activities and are concentrated mainly in metropolitan areas.

Any alteration to air composition can be defined as air pollution. In particular, substances which cause direct damage to human health are particulate matter (particulate of less than 10 millionths of a meter in diameter, PM10, and smaller than 2.5 μm, PM2.5). These substances are mainly concentrated in metropolitan areas. Traffic, industrial plants and the heating of buildings are just some of the causes of the continuous exceeding of the imposed limits.

The increasing public awareness on air pollution is making monitoring measures which are more representative of the real state of the urban environment more current and necessary. New environmental regulations are also designed to increase local pollutant measurements.

Air quality typically varies greatly in urban spaces as it is influenced by multiple parameters, such as: weather variables, traffic intensity and complex urban terrain. For example, precise sources of emissions are generally present in commercial and industrial areas, while generally widespread sources are present in residential areas. Therefore, in order to adequately characterize the areas of interest, it is necessary to:
monitor with a large number of stations appropriately distributed throughout the urban area;
intensify the number of sensors to produce maps of pollutant concentrations in urban and suburban areas with a high space resolution.

These conditions thus limit the maximum installation distance between the nodes of a monitoring station network.

Conventional monitoring systems have two major limitations:
do not allow a real-time pollutant map to be obtained as they collect data on a time scale of the order of days, and
they are often designed to monitor a single pollutant with large footprints, which are not compatible with massive installations.

The purpose of this invention is to provide a method and respective apparatus for tracking gaseous pollutants and mapping the sources of pollution in a given geographical area, thus certifying all air quality data generated by the monitoring network in terms of time and space.

More specifically, it is the object of the invention to provide a method and respective apparatus for monitoring air quality by sending real-time data on both pollutant concentrations and weather parameters with a much higher space-time resolution than current conventional technologies, thus allowing a real tracking of both gaseous and powder pollutants, and a direct identification the sources of pollution.

This result is achieved:

by providing a new type of air quality monitoring station configured to pre-process air quality data together with weather data, to store the data in its own database and to send the data together with its GPS coordinates to a central collection and processing computer, on which the data received from each station is classified and recorded;

by establishing, with a number of such stations, a network of monitoring stations said to have high space resolution (i.e. with a maximum distance between stations of between one and three kilometers in a straight line), in which each node in the network stores a copy of the database and updates it, being a monitoring station itself, and in which each station is positioned at the vertex of a number of adjacent triangles into which the geographical area to be mapped is divided;

and by equipping said central data collection and processing computer with an algorithm, configured to process information in real time and to supply the positions of the sources of pollution directly.

This thorough distribution is paired with a data transmission frequency having a high time resolution (between one and 10 minutes) in order to create a practically constant data flow which allows to micro-map the pollution sources also over time.

Specific solutions can therefore be found to either reduce or eliminate the identified sources of pollution. The data, once processed and stored in the general database, can be viewed with a computer platform, which can display the simplified representation of the monitoring data with different processing levels in real time.

By managing data using blockchain-based technology, it is permanently written in the distributed database. This allows to certify all air quality data generated by the monitoring network to be in both spatial and temporal terms.

According to an advantageous feature of the invention, the innovative monitoring station allows to identify three categories of environmental parameters in particular: weather, gaseous concentrations, and particulate matter concentrations.

The set of measured weather parameters essentially consists of: wind direction and intensity, temperature, relative humidity, atmospheric pressure. Conversely, the set of gaseous concentrations essentially consists of: carbon monoxide, ozone, nitrogen dioxide, hydrogen sulfide, sulfur dioxide, methane, ammonia. The parameters of particulate matter are essentially: PM 10, PM 2.5 and PM 1.

Further features and advantages of the invention will be more apparent in light of the detailed description which follows, making reference to the accompanying drawings which illustrate a preferred embodiment by way of non-limiting example. On the drawings:

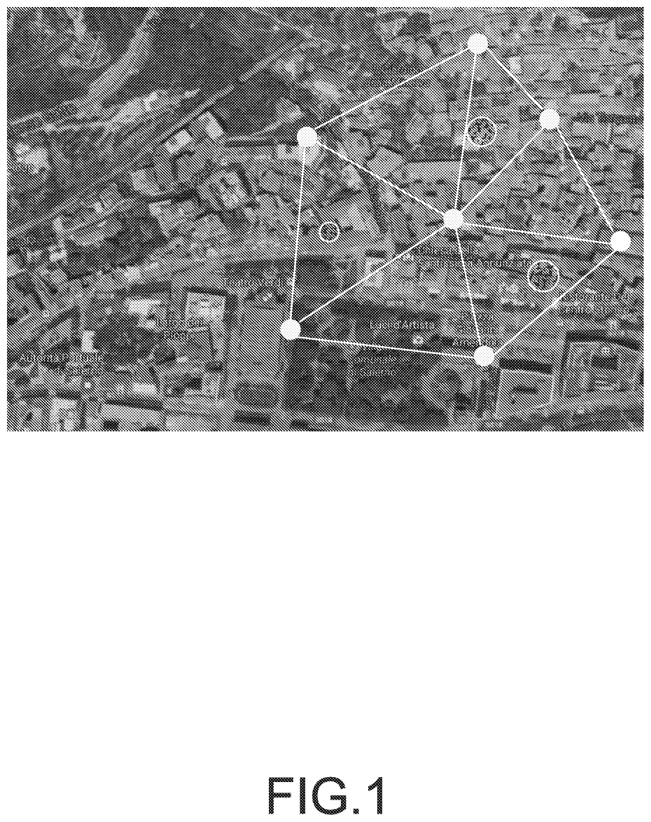

FIG. 1 is a diagram of a monitoring network according to the invention;

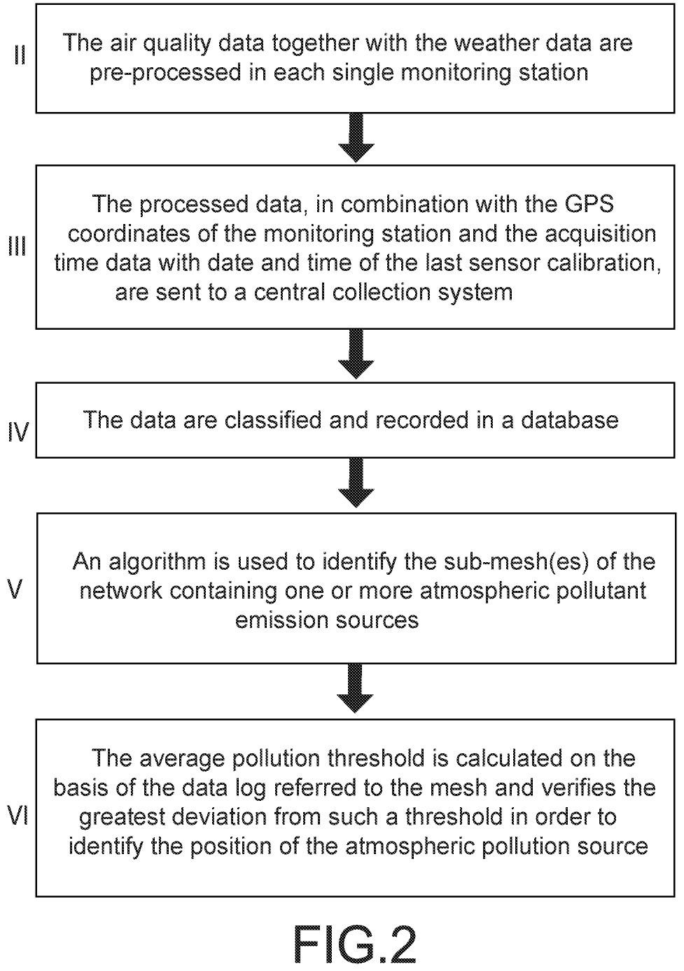

FIG. 2 is a block chart of the operation of the monitoring network itself;

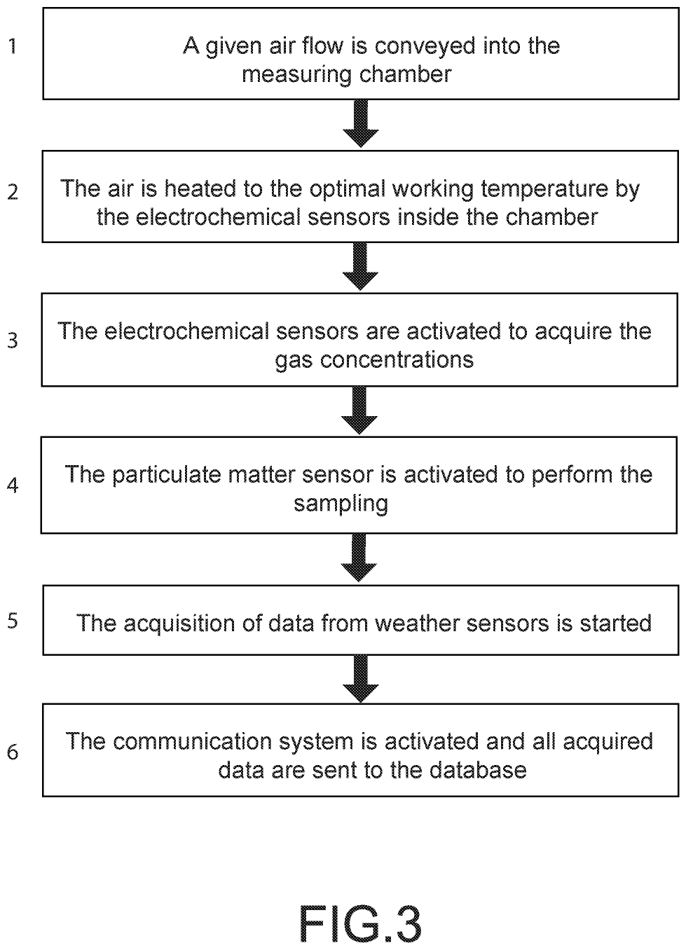

FIG. 3 is a block chart of the operation of the sensors used in each station;

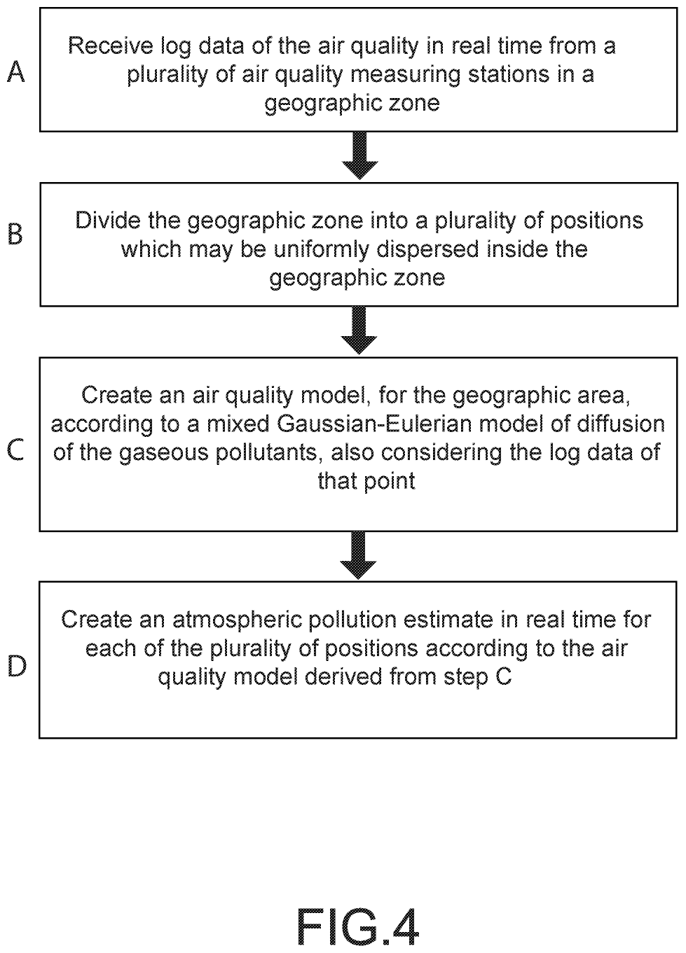

FIG. 4 is a block chart which shows the sequence of operating steps which are performed by the data collection and processing center according to the invention on the basis of an algorithm configured for the purpose;

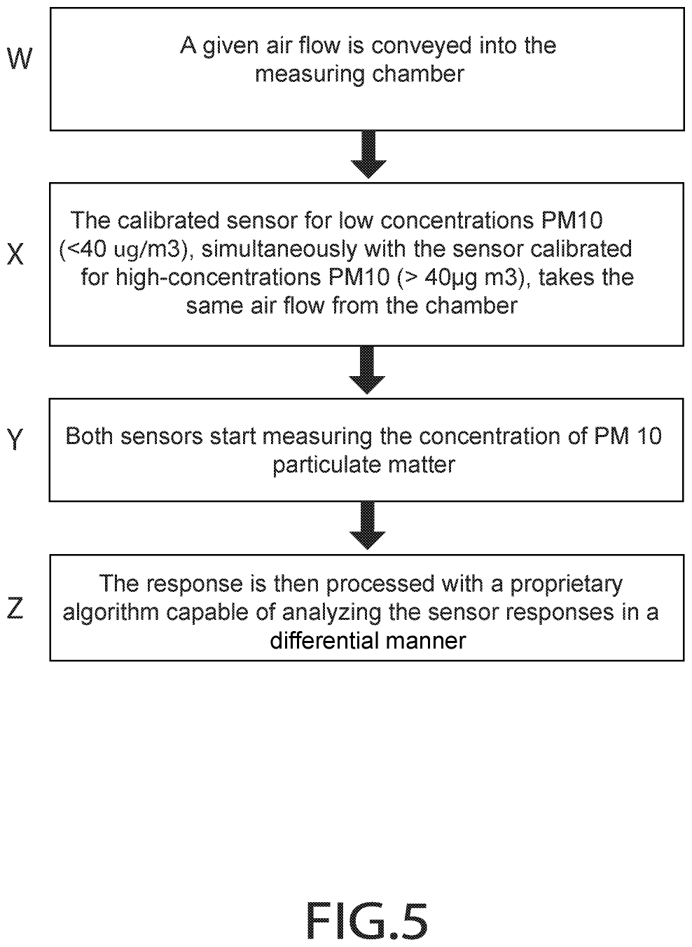

FIG. 5 is a block chart of the operative steps of the particulate matter measuring system;

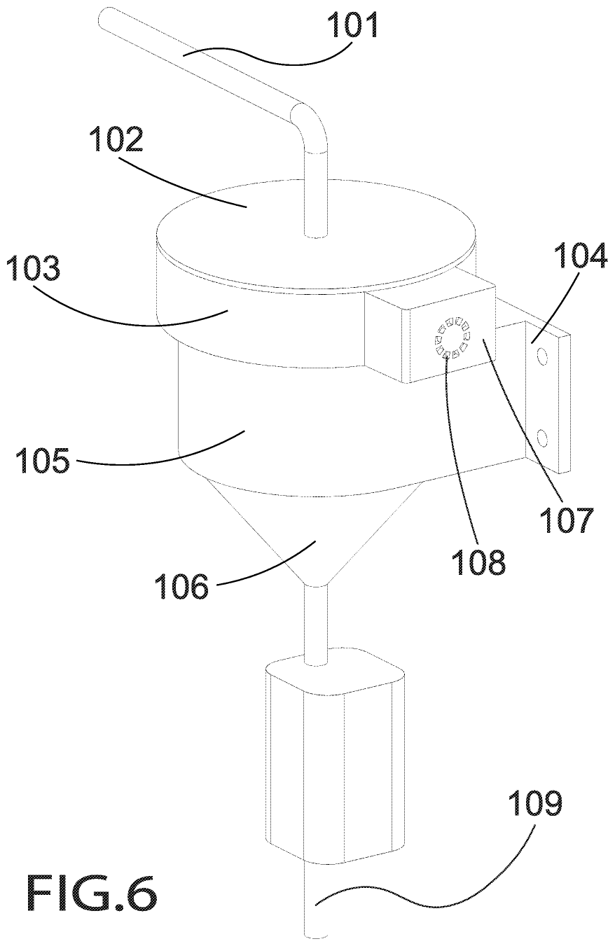

FIG. 6 is a perspective view of a monitoring system according to the invention;

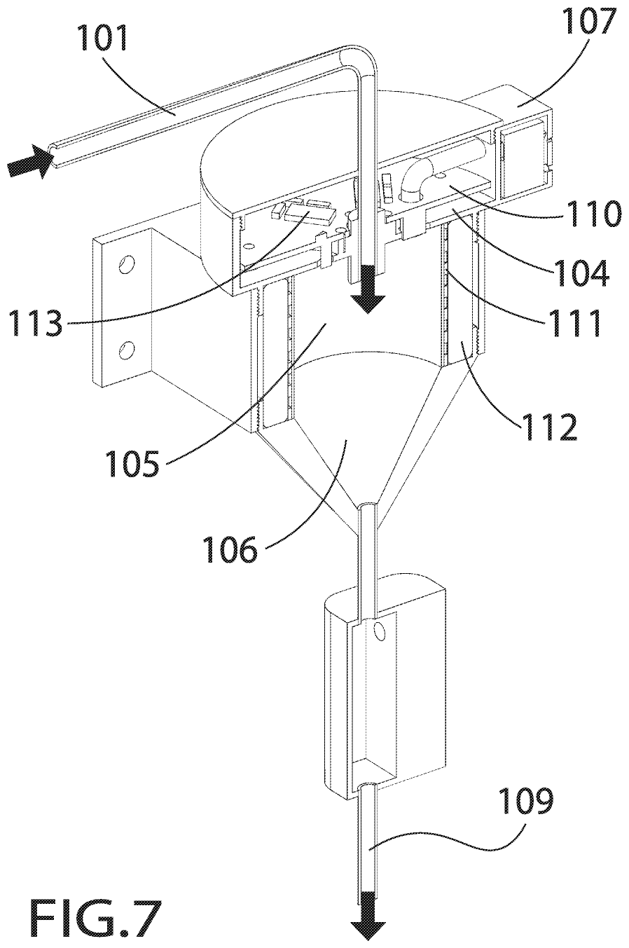

FIG. 7 is a perspective view of a vertical section of the monitoring station according to the invention, which shows the path of the air drawn into the station itself by a suction pump;

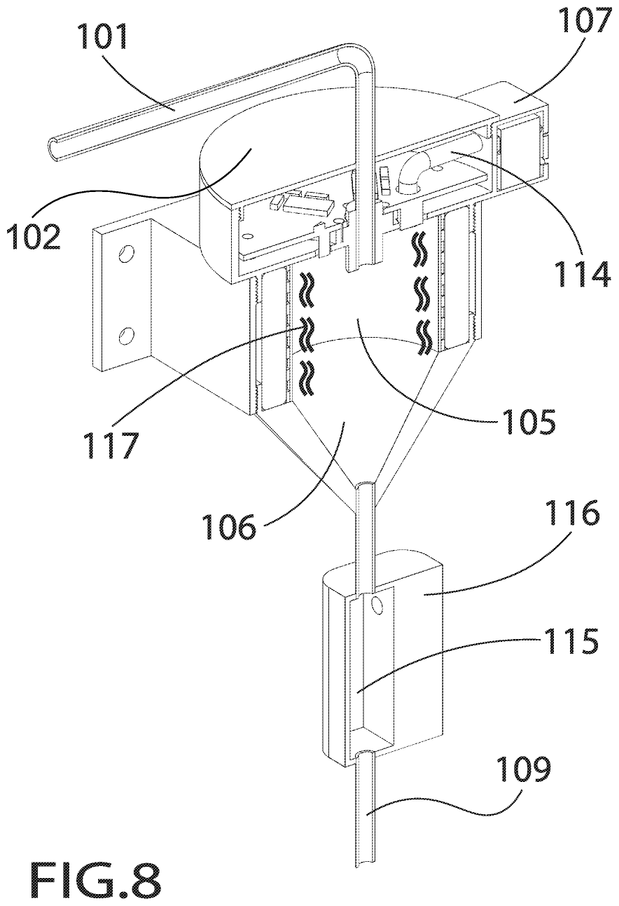

FIG. 8 is a perspective view of a vertical section of the monitoring station according to the invention, which shows the heating system of the measuring chamber;

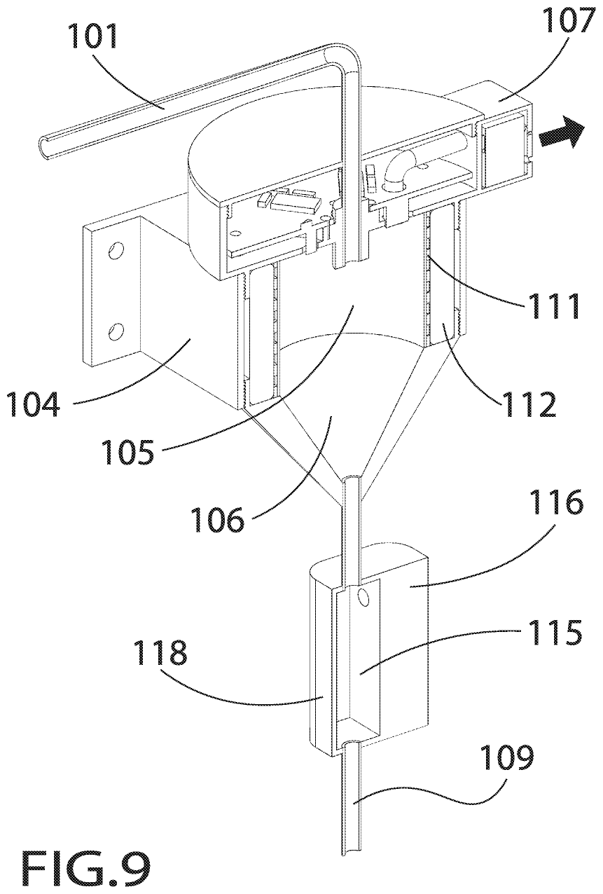

FIG. 9 is a perspective view of a vertical section of the monitoring station which shows the path of the air for measuring particulate matter;

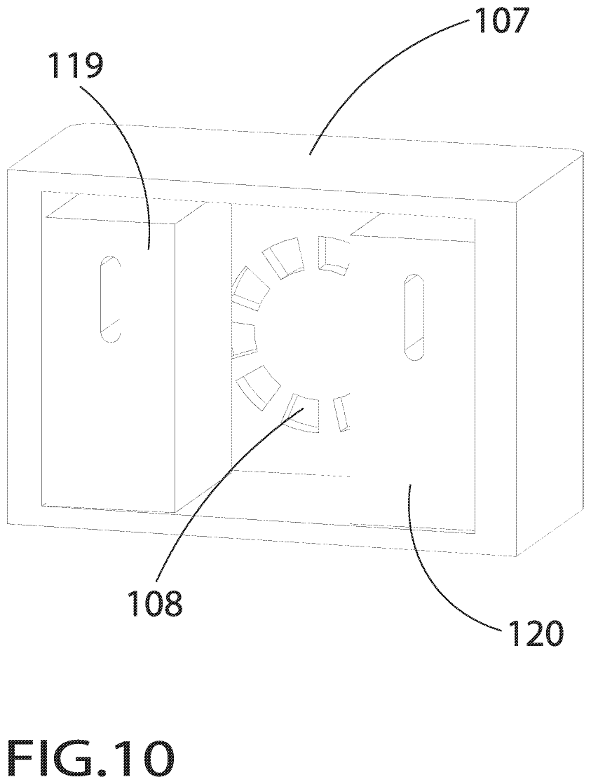

FIG. 10 is a perspective view of the particulate matter measuring system;

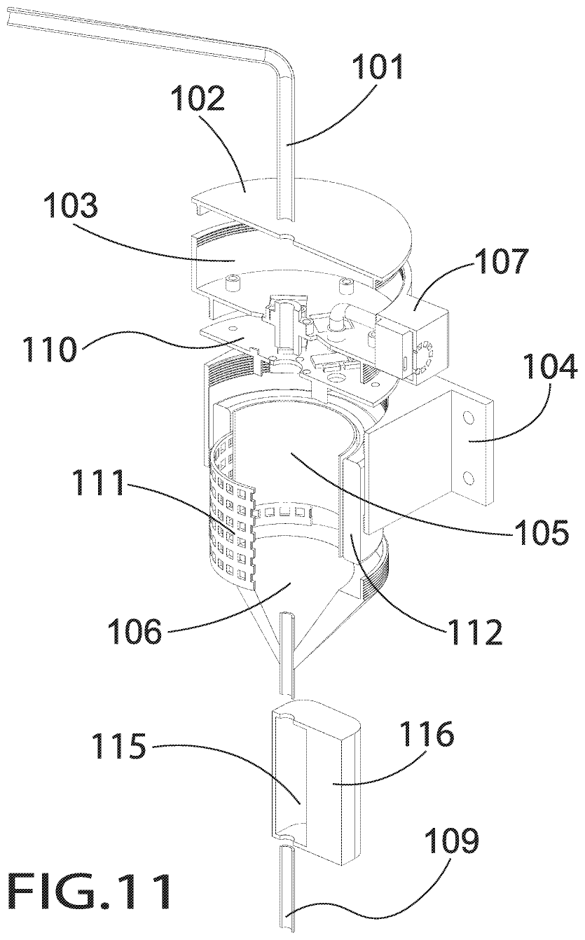

FIG. 11 is an exploded perspective view of the monitoring station in vertical section;

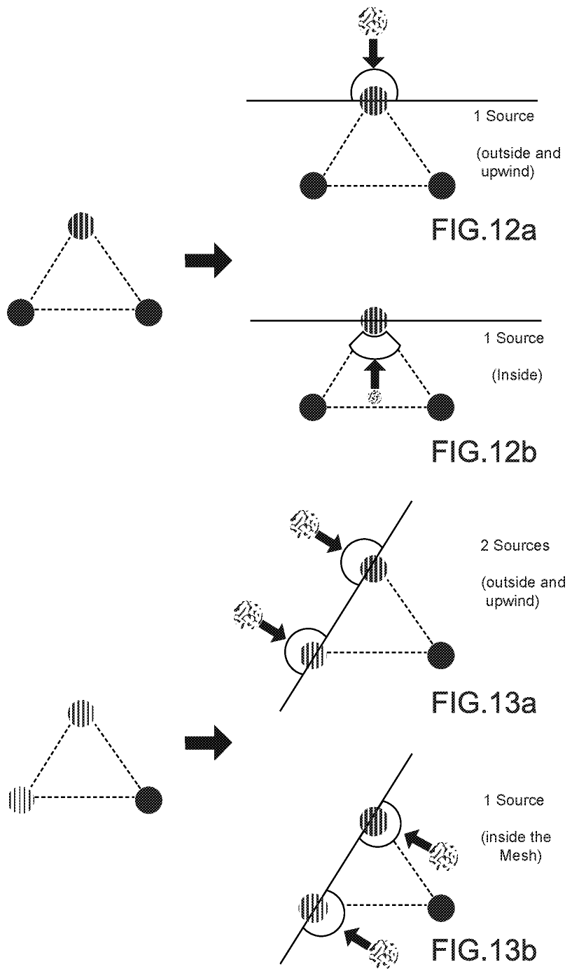

FIGS. 12a, 12b graphically shows the case of pollution indication of a single station of a mesh;

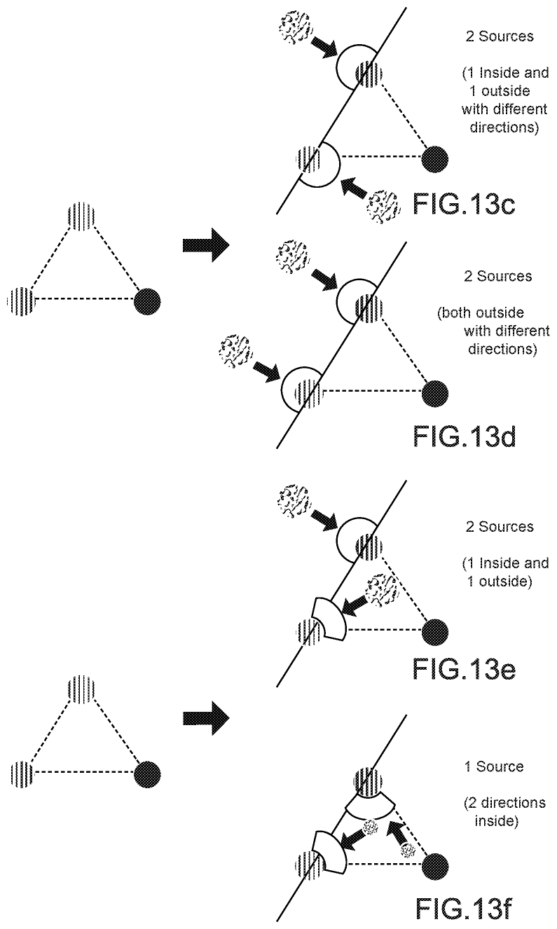

FIGS. 13a, 13b, 13c, 13d, 13e and 13f graphically show, in the case of signaling from two monitoring stations in the same mesh, the various possible situations according to the direction of the wind and the position of the polluting source, whether inside or outside the mesh itself;

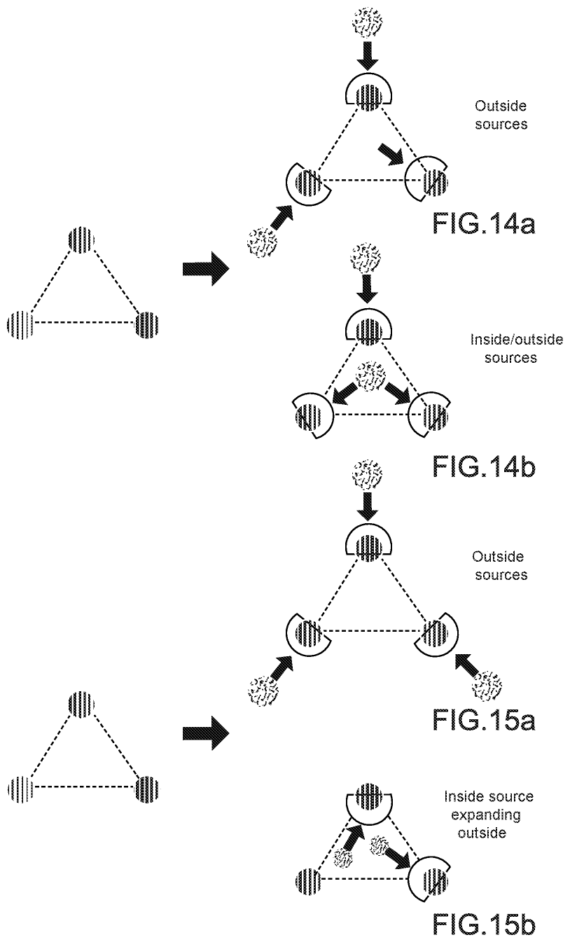

FIGS. 14a, 14b and 15a, 15b diagrammatically show the case of pollution indication coming from three stations of the same mesh, according to the wind direction and to the position of the sources themselves, whether inside or outside the mesh.

### DETAILED DESCRIPTION OF THE INVENTION

### Apparatus

A fundamental element of the invention is the monitoring station, which is shown in FIGS. 6 to 11. The station comprises a measuring chamber consisting of an upper cylindrical chamber 105 and a lower conical portion 106. An air inlet pipe 101 which passes through an upper circular compartment 103 which is closed by a removable cover 102 is attached to the upper cylindrical chamber 105, which is equipped with a fixing bracket 104 for its positioning. Said compartment 103 contains (FIG. 7) one or more electrochemical sensors 113, for measuring the concentrations of the polluting species present in the air, positioned on an inner shelf 110, above the base plane 104 which constitutes the ceiling of said chamber 105.

The compartment 103 is flanked by a box-like container 107 containing the particulate matter concentration measuring system. The particulate matter is taken, through a manifold duct 114 (FIG. 8), from the chamber 105 into which they are drawn by an air suction apparatus, which is inside a container 118 (FIG. 9) positioned at the lower end of the conical chamber 106. Said suction apparatus comprises a compartment 115, connected on the one side to the analyzed air expulsion tube 109 and on the other to a suction pump 116.

As shown in FIGS. 6 and 10, the particulate matter measuring system, inside the box-like container 107, comprises two different sensors 119, 120 based on the laser diffraction principle, which are positioned on opposite sides of a rosette 108 consisting of a plurality of air expulsion holes. Said sensors 119 and 120 simultaneously measure the same sample of air aspired from the chamber 105 by the suction pump 116 and by measuring the deviation between the two measured values it is possible to obtain a more accurate response, the measurer 119 being calibrated for low concentrations PM10 (<40 μg m³), while the other measurer 120 is calibrated for the high concentrations PM10 (>40 μg m³). This allows a differential response of the sensor system.

According to a particular feature of the invention, the cylindrical chamber assembly 105 and the conical portion 106 are used to stabilize and control the sampled air parameters. Indeed, it allows to sample the air even under unfavorable temperature and humidity conditions, by virtue of the presence of an inner heating system (FIG. 11) which comprises a heating wall 111 which surrounds the inner wall of the entire chamber 105 and offers the possibility of regulating the temperature using a quantity of energy regulated by an algorithm which optimizes its use for heating.

Advantageously, in order to reduce heat loss, the outer wall 112 of the cylindrical chamber 105 is made of an insulating material of appropriate thickness.

The sampling procedure of the monitoring station described above basically consists of the following steps, as shown in the block chart in FIG. 3:
1) The pump 116 aspirates air into chamber 105.
2) The air is heated inside chamber 105 through the heated cylindrical walls 111.
3) The reading of the electrochemical sensors is started to determine the concentration of the polluting gas species in chamber 105.
4) The concentration of the particulate matter which has entered the container 107 is measured.
5) The acquisition of data from weather sensors is started.
6) The data communication system is activated and the data are sent to the database of the central data collection and processing computer.

In order to allow a correct measurement of the concentration of the pollutants present, it is necessary to control the air flow rate at which the analysis is performed precisely. Through the pump 116, the particulate matter measuring system can vary the sampling speed and to adapt the inlet flow rate of the polluted air to said speed.

The block chart of the operation of the particulate matter measuring system is shown in FIG. 4.

The following operating steps are included:

Block A: A given air flow is conveyed into the measuring chamber.

Block B: The calibrated sensor for low concentrations PM10 (<40 μg m³) takes the same air flow from the chamber at the same time as the sensor calibrated for high concentrations PM10 (>40 μg m³).

Block C: Both sensors start measuring the concentration of PM 10 particulate matter.

Block D: The response is processed by the monitoring station itself by implementing an algorithm which can analyze the responses of the sensors in differential manner.

The node is engineered to send the measurements with the various wireless connection technologies (WIFI, GPRS and LORA) and via wire (Ethernet, serial) with a high time resolution, in particular, the sampling frequency can be set up to one measurement per minute, thus obtaining a huge quantity of data.

The network node was designed to be easy to install, and, by sending data in fully autonomous manner, it requires minimal configuration by the installer.

a computer platform was developed, which simplifies the use of the data, in order to make the monitoring data available to citizens 24 hours a day.

### Method

In order to map the sources of pollution and track the pollutants present, the inventors started from the observation that in order to increase pollution monitoring effectiveness it is necessary to identify the point or points from which a pollutant is emitted into the atmosphere with a reasonable approximation. Only in this way will it be possible to assign responsibility and suggest the corrective measures to be taken.

In a city, or industrial context, positioning the emission source with a certain approximation is a difficult task. This is because there are a number of potential sources of pollution which can also be precise in nature (e.g. the chimney of an industrial plant) or widespread (e.g. vehicle traffic smog).

An additional complication is represented by wind and urban morphology, which together create cells for mixing pollutants, making it even more difficult to identify the primary source of pollution.

In a first embodiment, the method of the present invention provides for:

I) Mapping the territory to be controlled (FIG. 1) with a series of monitoring stations (assuming a distribution of one station every 1-3 km²) having the features defined above, dividing it into many adjacent triangles (having one side of the triangle in common, i.e. two sensors). Each triangle of the network will be named “mesh” hereinafter for the sake of simplicity;

Block II (FIG. 2) Evaluating the air quality data in each monitoring station, i.e.:
the wind intensity and direction;
the relative humidity;
the atmospheric pressure;
the concentration of gaseous pollutants and pre-processing the air quality data together with the weather data in each single monitoring station; autonomous processing means are provided in each monitoring station for this purpose.

Block III) The processed data, in combination with the GPS coordinates of the monitoring station and the acquisition time data with date and time of the last sensor calibration, are sent to a computerized collection and processing center.

Block IV) The data are classified and recorded in a database by said collection center.

Block V) The data collection and processing center, by implementing an algorithm, identifies the mesh(es) of the network containing one or more sources of atmospheric pollutants.

Block VI) Calculates the mean pollution threshold on the basis of the data log referred to the mesh and verifies the greatest deviation from such a threshold in order to identify the position of the atmospheric pollution source.

According to whether the increase in concentration of the pollutant is identified by none, one, two or all three monitoring stations of a mesh, different situations are identified as shown in FIGS. 12-15, in which the graphic symbols used are as follows:

[symbol]

Pollution source [symbol]
Monitoring station with pollutant variation indication [symbol]
Monitoring solution no variation of pollutants
⬇ [symbol]
Wind direction
In particular, FIG. 12 shows the case in which only one of the three stations indicates an increase in concentration of the pollutant.

Two alternatives may occur in this case:

If the wind comes from outside the mesh (FIG. 12a), it is apparent that the pollution increase is due to a source outside the mesh, in particular located in the direction opposite to the wind direction.

If the wind comes from a direction inside the mesh (FIG. 12b), then the pollution increase is due to a source inside the mesh.

FIG. 13 shows the case in which two of the three stations of a same mesh indicate an increase in concentration of the pollutant.

Four different situations may occur in this case:

If the wind of the two stations in which the concentrations increase comes from the same direction, and in particular from the opposite half-plane of the mesh (FIG. 13a), it can be concluded that the pollution increase is due to a single source outside the mesh, particularly located in the direction opposite to the wind direction.

If the wind of the two stations in which the concentrations increase comes from the same direction and in particular from the half-plane of the mesh

(FIG. 13b) the increase of pollution is certainly due to a single source inside the mesh.

If the wind of the two stations in which the concentrations increase comes from two different directions and in particular one from the half-plane of the mesh (FIG. 13c) and the other from the half-plane opposite to the mesh, then it is apparent that the pollution increase is due to two sources of pollution, one outside the mesh and the other inside.

If the wind of the two stations in which the concentrations increase comes from two different directions and in particular both from outside the mesh (FIG. 13d), then the pollution increase is due to two sources of pollution, both outside the mesh.

If the wind of the two stations in which the concentrations increase comes from two different directions and in particular one from outside the mesh and one from inside the mesh (FIG. 13e), then the pollution increase is due to two sources of pollution, one outside and the other inside the mesh.

If the wind of the two stations in which the concentrations increase comes from two different directions but both inside the mesh (FIG. 13f), then the pollution increase is due to a single source of pollution inside the mesh.

Other cases with three sources are shown by way of example in FIGS. 14a, 14b and 15a, 15b.

In a second embodiment, (FIG. 5) the processing center to which the data from the various measuring stations refer, is configured to:

Block W: Receive log data of the air quality in real time from a plurality of air quality measuring stations in a given geographic zone.

Block X: Divide the geographic zone into a plurality of positions which may be uniformly dispersed inside said geographic zone.

Block Y: Create an air quality model, for the geographic area, according to a mixed Gaussian-Eulerian model of diffusion of the gaseous pollutants, also considering the log data of that point. This will autonomously allow the single station to send possible warnings that the levels of pollutants have been exceeded.

Block Z: Create an atmospheric pollution estimate in real time for each of the plurality of positions according to the air quality model derived from step Y and from the air quality data in real time.

An apparatus and preferred embodiments of the method according to the invention have been described hereto. It is finally apparent that many changes and variations can be made without departing from the scope of protection defined by the appended claims.

The invention claimed is:

1. A monitoring station for concentrations of pollutants and particulate matter and for local atmospheric and weather parameters, comprising:
a measuring chamber configured to be filled with air to be analyzed;
means for controlling a polluted air flow rate entering the measuring chamber and to be analyzed;
means for heating the air contained inside said measuring chamber so as to adjust a temperature of sampled air even under adverse temperature and humidity conditions;
sensors which allow measuring the concentrations of pollutants present in the air;
a particulate matter measuring assembly, based on laser diffraction and comprising two detectors, one of the two detectors being calibrated for low concentrations and one of the two detectors being calibrated for high concentrations, the two detectors simultaneously measuring a same air sample aspirated into said measuring chamber, so that the sensors provides a differential response;
means for evaluating wind direction and intensity, temperature, relative humidity, and atmospheric pressure in an environment surrounding the monitoring station;
a processor and an algorithm which can be implemented with said processor, the processor and the algorithm being configured to pre-process detected air quality data in combination with detected weather data to generate processed data;
means for storing and sending the processed data, in combination with GPS coordinates and acquisition time data, with date and time of last sensor calibration, to a computerized collection and processing center, using a wireless connection having high time resolution;
means for varying a sampling frequency of the monitoring station; and
an autonomous energy source adapted to avoid all electrical connections by an installer.

2. The monitoring station according to claim 1, wherein the air is recalled into the measuring chamber by a suction pump contained at an end of a conical manifold underneath said measuring chamber.

3. The monitoring station according to claim 1, further comprising means for identifying the GPS coordinates of a place where the monitoring station is positioned.

4. The monitoring station according to claim 1, wherein the sensors are electrochemical sensors configured to detect gaseous concentrations of pollutants.

5. The monitoring station according to claim 1, wherein the two detectors are calibrated for PM10, PM2.5 and PM1.

6. An apparatus for mapping sources of atmospheric pollution and tracking pollutants, comprising:
a network of monitoring stations having high spatial resolution, the high spatial resolution being an ability to measure a maximum distance between the monitoring stations from one to three kilometers in a straight line, wherein each monitoring station is a station according to claim 1 and is positioned at a vertex of a plurality of adjacent triangles or meshes, into which a geographic zone to be mapped is divided;
a central data collection and processing computer configured to receive pre-processed data from each of said monitoring stations, and classify and record the pre-processed data in a database to build a log, and an algorithm adapted to be implemented on said central data collection and processing computer, to process said data from each of the monitoring stations in real time, and directly provide positions of pollution sources to compare said positions with a data log from a previous mesh in the database, thus verifying a greatest deviation from a mean threshold.

7. The apparatus according to claim 6, wherein a sending frequency of high time resolution data is from one to 10 minutes, so as to create an essentially constant data flow which allows micro-mapping the pollution sources over time.

8. The apparatus according to claim 6, further comprising a computerized platform designed to display the data processed and stored in the database, in order to provide a simplified representation of monitoring data in real time and with different processing levels.

9. The apparatus according to claim 6, wherein the data of the network of monitoring having high space and time resolution are stored in a distributed database, while each node of the network stores a copy of the distributed database and updates the distributed database, the node being a monitoring station.

10. The apparatus according to claim 9, wherein the data are managed using blockchain-based technology and are permanently written in the distributed database, thus allowing certifying all air quality data generated by the network in terms of time and space, and wherein the nodes of the network, in addition to monitoring air quality, are configured to enter data into the distributed database, thereby validating data of other nodes.

11. A method of mapping polluting sources and tracking pollutants by using monitoring networks comprising stations according to claim 1, comprising the steps of:
mapping a territory to be controlled with monitoring stations according to claim 1, the monitoring stations being distributed every 1-3 km, dividing the territory into adjacent triangles or meshes sharing one side, so as to share two sensors;
evaluating in each monitoring station:
wind intensity and direction;
relative humidity;
atmospheric pressure;
concentration of gaseous pollutants and particulate matter;
pre-processing air quality data together with weather data in each monitoring station;
sending the processed data, in combination with GPS coordinates of the monitoring station and acquisition time data with date and time of a last sensor calibration, to a computerized collection and processing center;
recording and classifying said processed data in a database of said collection and processing center;
identifying in said collection and processing center, by implementing an algorithm, the meshes of a network containing one or more sources of the pollutants in an atmosphere; and
calculating, in the computerized collection and processing center, a mean pollution threshold based on data log referred to the mesh and verifying a greatest deviation from the mean pollution threshold in order to identify a position of an atmospheric pollution source.

12. The method according to claim 11, further comprising the step of measuring an increase in pollutant concentration with one, two, or three mesh monitoring stations.

13. The method according to claim 12, wherein two alternatives may occur if only one of three stations indicates an increase in pollutant concentration:
if wind comes from outside of the mesh, then the increase in the pollutant concentration is attributed to a source outside of the mesh; or
if the wind comes from a direction inside of the mesh, then the increase in the pollutant concentration is attributed to a source inside of the mesh.

14. The method according to claim 12, wherein four different situations are identified if two of three stations of same mesh indicate an increase the in pollutant concentration:
a) if wind direction indicated by the two stations, in which an increase in the pollution concentration is detected, is in a direction opposite to a half-plane of the mesh, the pollution increase is due to a single source outside of the mesh;
b) if the wind direction indicated by the two stations, in which an increase in the pollution concentration is detected, is in a direction integral with the half-plane of the mesh, the pollution increase is attributed to a single source inside the mesh;
c) if the wind of the two stations, in which an increase in the pollution concentration is detected ,comes from two different directions, then the pollution increase is attributed to two sources of pollution, one outside of the mesh and another one inside of the mesh;
d) if the wind of the two stations, in which an increase in the pollution concentration is detected, comes from two different, then the pollution increase is attributed to two sources of pollution, both outside of the mesh;
e) if the wind of the two stations in which the concentrations increase comes from two different directions and in particular one from outside the mesh and one from inside the mesh, then the pollution increase is attributed to two sources of pollution, one being outside and the other inside the mesh; and
f) if the wind of the two stations, in which an increase in the pollution concentration is detected, comes from two different directions, both inside of the mesh, then the pollution increase is attributed to a single source of pollution inside the mesh.

15. The method according to claim 11, wherein the collection and processing center, to which the data from different measuring stations refer, is provided with an algorithm configured to:
receive log data of the air quality in real time from a plurality of the monitoring stations in a geographic zone (Block W);
divide the geographic zone into a plurality of positions that are uniformly dispersed inside said geographic zone (Block X);
create an air quality model, for the geographic zone, according to a mixed Gaussian-Eulerian model of diffusion of the pollutants, considering the log data, so that a single station to send possible is enabled to send warnings when predetermined levels of the pollutants have been exceeded (Block Y); and
create an atmospheric pollution estimate in real time for each of the plurality of positions according to the air quality model and from air quality data in real time (Block Z).

16. The monitoring station according to claim 1, further comprising a node where a concentration of specific pollution elements are measured.

17. The monitoring station according to claim 16, wherein the node measures air density and measures the concentration of PM2.5 and PM10 with greater precision than a scattering precision, and wherein residual dusts captured in filters is measured, measuring chemical elements in particulate matter.
